SEQUENCE LISTING (12 sequences)

SEQ ID NO: 1 - DNA, 30 bp, synthetic construct
  misc_feature 1..30: note = synthetic polymer
  source 1..30: mol_type = other DNA
ccctcagattcaactggcagtaaccagaat

SEQ ID NO: 2 - DNA, 30 bp, synthetic construct
  misc_feature 1..30: note = synthetic polymer
  source 1..30: mol_type = other DNA
aagatcacattggcacccgcaatcctgcta

SEQ ID NO: 3 - DNA, 30 bp, synthetic construct
  misc_feature 1..30: note = synthetic polymer
  source 1..30: mol_type = other DNA
aaattgcacaatttgcccccagcgcttcag

SEQ ID NO: 4 - DNA, 20 bp, synthetic construct
  misc_feature 1..20: note = synthetic polymer
  source 1..20: mol_type = other DNA
gaccccaaaatcagcgaaat

SEQ ID NO: 5 - DNA, 24 bp, synthetic construct
  misc_feature 1..24: note = synthetic polymer
  source 1..24: mol_type = other DNA
tctggttactgccagttgaatctg

SEQ ID NO: 6 - DNA, 22 bp, synthetic construct
  misc_feature 1..22: note = synthetic polymer
  source 1..22: mol_type = other DNA
gggagccttgaatacaccaaaa

SEQ ID NO: 7 - DNA, 21 bp, synthetic construct
  misc_feature 1..21: note = synthetic polymer
  source 1..21: mol_type = other DNA
tgtagcacgattgcagcattg

SEQ ID NO: 8 - DNA, 24 bp, synthetic construct
  misc_feature 1..24: note = synthetic polymer
  source 1..24: mol_type = other DNA
accccgcattacgtttggtggacc

SEQ ID NO: 9 - DNA, 24 bp, synthetic construct
  misc_feature 1..24: note = synthetic polymer
  source 1..24: mol_type = other DNA
aycacattggcacccgcaatcctg

SEQ ID NO: 10 - DNA, 19 bp, synthetic construct
  misc_feature 1..19: note = synthetic polymer
  source 1..19: mol_type = other DNA
agatttggacctgcgagcg

SEQ ID NO: 11 - DNA, 20 bp, synthetic construct
  misc_feature 1..20: note = synthetic polymer
  source 1..20: mol_type = other DNA
gagcggctgtctccacaagt

SEQ ID NO: 12 - DNA, 23 bp, synthetic construct
  misc_feature 1..23: note = synthetic polymer
  source 1..23: mol_type = other DNA
ttctgacctgaaggctctgcgcg

(19) United States
(12) Patent Application Publication
(10) Pub. No.: US 2023/0131612 A1
(43) Pub. Date: Apr. 27, 2023

(54) METHODS FOR DETECTION OF SEVERE ACUTE RESPIRATORY SYNDROME CORONAVIRUS 2

(71) Applicant: Avellino Lab USA, Inc., Menlo Park, CA (US)

(72) Inventors: Jun-Heok JANG, Menlo Park, CA (US); Connie CHAO-SHERN, Menlo Park, CA (US)

(21) Appl. No.: 17/895,848

(22) Filed: Aug. 25, 2022

Related U.S. Application Data
(63) Continuation of application No. PCT/US2021/016245, filed on Feb. 2, 2021.
(60) Provisional application No. 63/022,984, filed on May 11, 2020.
(60) Provisional application No. 63/011,204, filed on Apr. 16, 2020.
(60) Provisional application No. 63/010,534, filed on Apr. 15, 2020.
(60) Provisional application No. 62/983,570, filed on Feb. 28, 2020.

(51) Int. Cl. C12Q 1/70

(52) U.S. Cl. CPC C12Q 1/701

(57) ABSTRACT

Methods and reagents for detection of severe acute respiratory syndrome coronavirus 2 (SARS-CoV 2) are described. Also described are methods and reagents for multiplexed detection of SARS-CoV 2 and methods and reagents for high-throughput detection of SARS-CoV 2.

### RELATED APPLICATIONS

This application is a continuation application of PCT Application No. PCT/US2021/016245, filed Feb. 2, 2021, which claims the benefit of, and priority to, U.S. Provisional Application No. 63/022,984, filed May 11, 2020, U.S. Provisional Application No. 63/011,204, filed Apr. 16, 2020, U.S. Provisional Application No. 63/010,534, filed Apr. 15, 2020, and U.S. Provisional Application No. 62/983,570, filed Feb. 28, 2020, each of which is incorporated by reference in herein in its entirety.

### FIELD OF THE APPLICATION

This application generally relates to methods for detection of coronavirus, in particular, severe acute respiratory syndrome coronavirus 2 (SARS-CoV-2).

### BACKGROUND

Severe acute respiratory syndrome coronavirus 2 (SARS-CoV 2), associated with COVID-19, has raised concerns around the world. The ability to diagnose patients with the virus plays a critical role in managing and controlling the global spread of the virus. With the rapid spreading of the SARS-CoV 2, there is a need for an increased capacity to test subjects.

### SUMMARY

On Jan. 24, 2020, Centers for Disease Control and Prevention has announced a test panel for detection of SARS-CoV 2 (the “CDC test panel”). The reagents and methods described herein have fewer false negative results compared to the CDC test panel.

In addition, detection of SARS-CoV 2 requires requires detection of the presence of multiple nucleotide sequences. Thus, testing of a single sample for the presence of SARS-CoV 2 requires separating the sample into multiple containers (e.g., PCR tubes) so that respective nucleotide sequences are detected in separate containers. The reagents and methods described herein have allow multiplexed detection of nucleotide sequences associated with SARS-CoV 2 in a single container. This allows reduction of reagents required for testing each sample and also increases the number of samples that can be tested simultaneously on a same sample plate, thereby increasing the throughput of SARS-CoV2 testing.

Furthermore, a pooled batch of samples from multiple subjects can be tested to determine that none of the samples from the multiple subjects contains SARS-CoV 2. For example, samples from 10, 20, 30, 40, 50, 60, 70, 80, 90, 100, or 120 subjects may be combined into a single batch, and testing the single batch for presence or absence of SARS-CoV 2 can be faster and more efficient than testing the samples from individual subjects separately (which would require 10, 20, 30, 40, 50, 60, 70, 80, 90, 100, or 120 tests or more). Moreover, testing of the pooled batch of samples may be performed using the multiplex detection method described herein, which enables testing of thousands, or tens of thousands of, samples on a single plate, thereby enabling high-throughput detection of SARS-CoV 2.

In accordance with some embodiments, a method for detecting SARS-CoV 2 in a sample from a subject includes determining presence of a first nucleotide sequence in a sample in a particular container using a first amplification primer pair; and determining presence of a second nucleotide sequence in the sample in the particular container using a second amplification primer pair.

In accordance with some embodiments, a method for detecting SARS-CoV 2 in a sample from a subject includes determining presence of a first nucleotide sequence in a pooled batch of samples including the sample from the subject (and samples from one or more other subjects) using a first amplification primer pair; and determining presence of a second nucleotide sequence in the pooled batch of samples using a second amplification primer pair. Conversely, in accordance with some embodiments, a method for determining absence of SARS-CoV 2 in a sample from a subject includes determining absence of a first nucleotide sequence in a pooled batch of samples including the sample from the subject (and samples from one or more other subjects) using a first amplification primer pair; and determining absence of a second nucleotide sequence in the pooled batch of samples using a second amplification primer pair.

In some embodiments, the pooled batch of samples includes samples from at least two subjects. For example, the pooled batch of samples may include samples from 2, 3, 4, 5, 6, 7, 8, 9, 10, 20, 30, 40, 50, 60, 70, 80, 90, 100, or 120 subjects or more, including ranges between any of the foregoing values.

In some embodiments, the first nucleotide sequence has at least 80% identity to SEQ ID NO: 1. In some embodiments, the first nucleotide sequence has at least 85%, 90%, 91%, 92%, 93%, 94%, 95%, 96%, 97%, 98%, or 99% identity to SEQ ID NO: 1. In some embodiments, the first nucleotide sequence is SEQ ID NO: 1.

In some embodiments, the second nucleotide sequence has at least 80% identity to SEQ ID NO: 2. In some embodiments, the second nucleotide sequence has at least 85%, 90%, 91%, 92%, 93%, 94%, 95%, 96%, 97%, 98%, or 99% identity to SEQ ID NO: 2. In some embodiments, the second nucleotide sequence is SEQ ID NO: 2.

In some embodiments, the third nucleotide sequence has at least 80% identity to SEQ ID NO: 3. In some embodiments, the third nucleotide sequence has at least 85%, 90%, 91%, 92%, 93%, 94%, 95%, 96%, 97%, 98%, or 99% identity to SEQ ID NO: 3. In some embodiments, the third nucleotide sequence is SEQ ID NO: 3.

In some embodiments, the first amplification primer pair includes an amplification primer having a nucleotide sequence having at least 80% identity to SEQ ID NO: 4. In some embodiments, the nucleotide sequence has at least 85%, 90%, 91%, 92%, 93%, 94%, 95%, 96%, 97%, 98%, or 99% identity to SEQ ID NO: 4. In some embodiments, the nucleotide sequence is SEQ ID NO: 4.

In some embodiments, the first amplification primer pair includes an amplification primer having a nucleotide sequence having at least 80% identity to SEQ ID NO: 5. In some embodiments, the nucleotide sequence has at least 85%, 90%, 91%, 92%, 93%, 94%, 95%, 96%, 97%, 98%, or 99% identity to SEQ ID NO: 5. In some embodiments, the nucleotide sequence is SEQ ID NO: 5.

In some embodiments, the second amplification primer pair includes an amplification primer having a nucleotide sequence having at least 80% identity to SEQ ID NO: 6. In some embodiments, the nucleotide sequence has at least 85%, 90%, 91%, 92%, 93%, 94%, 95%, 96%, 97%, 98%, or 99% identity to SEQ ID NO: 6. In some embodiments, the nucleotide sequence is SEQ ID NO: 6.

In some embodiments, the second amplification primer pair includes an amplification primer having a nucleotide sequence having at least 80% identity to SEQ ID NO: 7. In some embodiments, the nucleotide sequence has at least 85%, 90%, 91%, 92%, 93%, 94%, 95%, 96%, 97%, 98%, or 99% identity to SEQ ID NO: 7. In some embodiments, the nucleotide sequence is SEQ ID NO: 7.

In some embodiments, the presence of the first nucleotide sequence is determined also using a first probe having a nucleotide sequence having at least 80% identity to SEQ ID NO: 8. In some embodiments, the nucleotide sequence has at least 85%, 90%, 91%, 92%, 93%, 94%, 95%, 96%, 97%, 98%, or 99% identity to SEQ ID NO: 8. In some embodiments, the nucleotide sequence is SEQ ID NO: 8.

In some embodiments, the presence of the second nucleotide sequence is determined also using a second probe having a nucleotide sequence having at least 80% identity to SEQ ID NO: 9. In some embodiments, the nucleotide sequence has at least 85%, 90%, 91%, 92%, 93%, 94%, 95%, 96%, 97%, 98%, or 99% identity to SEQ ID NO: 9. In some embodiments, the nucleotide sequence is SEQ ID NO: 9.

In some embodiments, the first probe and the second probe have distinct labels.

In some embodiments, the method also includes determining presence of RNAse P gene in the sample in the particular container using a third amplification primer pair.

In some embodiments, the method also includes determining presence of RNAse P gene in the pooled batch of samples using a third amplification primer pair.

In some embodiments, the third amplification primer pair includes an amplification primer having a nucleotide sequence having at least 80% identity to SEQ ID NO: 10.

In some embodiments, the third amplification primer pair includes an amplification primer having a nucleotide sequence having at least 80% identity to SEQ ID NO: 11.

In some embodiments, presence of RNAse P gene in the sample is determined also using a third probe having a nucleotide sequence having at least 80% identity to SEQ ID NO: 12.

In some embodiments, presence of RNAse P gene in the pooled batch of samples is determined also using a third probe having a nucleotide sequence having at least 80% identity to SEQ ID NO: 12.

In some embodiments, the first probe, the second probe, and the third probe have distinct labels.

In some embodiments, the presence of RNAse P gene is detected by real-time reverse transcription polymerase chain reaction.

In some embodiments, the RNAse P gene is human RNAse P gene.

In some embodiments, the presence of the first nucleotide sequence is detected by real-time reverse transcription polymerase chain reaction while the sample is in the particular container.

In some embodiments, the presence of the second nucleotide sequence is detected by real-time reverse transcription polymerase chain reaction while the sample is in the particular container.

In some embodiments, the presence of the first nucleotide sequence is detected by real-time reverse transcription polymerase chain reaction while the pooled batch of samples is in a particular container.

In some embodiments, the presence of the second nucleotide sequence is detected by real-time reverse transcription polymerase chain reaction while the pooled batch of samples is in the particular container.

In some embodiments, the presence of the first nucleotide sequence and the presence of the second nucleotide sequence are detected by concurrent real-time transcription polymerase chain reaction.

In some embodiments, the presence of the RNAse P gene is detected by real-time reverse transcription polymerase chain reaction while the pooled batch of samples is in the particular container.

In some embodiments, the subject is a human.

In some embodiments, the method includes, in accordance with a determination that at least both the first nucleotide sequence and the second nucleotide sequence are present in the pooled batch of samples from multiple subjects, determining presence of SARS-CoV 2 in samples from the multiple subjects separately. For example, when a first pooled batch of samples from 72 subjects is determined to contain SARS-CoV 2, the samples from the subjects who contributed to the pooled batch of samples are tested separately without further pooled testing.

In some embodiments, the method includes, in accordance with a determination that both the first nucleotide sequence and the second nucleotide sequence are present in the pooled batch of samples from multiple subjects, determining presence of SARS-CoV 2 in a second batch of samples from a subset, less than all, of the multiple subjects. Similarly, in some embodiments, the method includes, in accordance with a determination that both the first nucleotide sequence and the second nucleotide sequence are present in the pooled batch of samples from multiple subjects, determining absence of SARS-CoV 2 in a second batch of samples from a subset, less than all, of the multiple subjects. For example, when a first pooled batch of samples from 72 subjects is determined to contain SARS-CoV 2, two separate batches (e.g., a second pooled batch containing samples from the first to thirty-sixth subjects and a third pooled batch containing samples from the thirty-seventh to the seventy-second subjects) are tested separately for detecting whether either or both batches contains SARS-CoV 2. If the second pooled batch is determined to contain SARS-CoV 2 but the third pooled batch is determined to be free from SARS-CoV 2, the samples from the subjects who contributed to the second pooled batch of samples may be splited into smaller batches (e.g., a fourth pooled batch containing samples from the first to eighteenth subjects and a fifth pooled batch containing samples from the nineteenth to thirty-sixth subjects) for further testing. Alternatively, the samples from the subjects who contributed to the second pooled batch of samples are tested separately without further pooled testing.

In some embodiments, the method includes, in accordance with a determination that the first nucleotide sequence, the second nucleotide sequence, and the RNAse P gene are present in the pooled batch of samples from multiple subjects, determining presence of SARS-CoV 2 in samples from the multiple subjects separately.

In some embodiments, the method includes, in accordance with a determination that the first nucleotide sequence, the second nucleotide sequence, and the RNAse P gene are present in the pooled batch of samples from multiple subjects, determining presence of SARS-CoV 2 in a batch of samples consisting of samples from a subset, less than all, of the multiple subjects.

In accordance with some embodiments, a method for determining absence of severe acute respiratory syndrome coronavirus 2 (SARS-CoV 2) in a sample from a subject includes determining that SARS-CoV 2 is absent in the sample in accordance with at least a determination that a first nucleotide sequence is absent in a pooled batch of samples including the sample and a determination that a second nucleotide sequence is absent in the pooled batch of samples. In some embodiments, the absence of the first nucleotide sequence in the pooled batch of samples and the absence of the second nucleotide sequence in the pooled batch of samples are concurrently determined in a same container (e.g., using multiplexed detection).

In accordance with some embodiments, a method for detecting severe acute respiratory syndrome coronavirus 2 (SARS-CoV 2) in a sample from a subject includes determining that SARS-CoV 2 is present in the sample in a particular container in accordance with at least a determination that a first nucleotide sequence is present in the sample and a determination that a second nucleotide sequence is present in the sample in the particular container. In some embodiments, the presence of the first nucleotide sequence in the sample in the particular container and the presence of the second nucleotide sequence in the sample in the particular container are concurrently determined.

In some embodiments, the first nucleotide sequence has at least 80% identity to SEQ ID NO: 1.

In some embodiments, the second nucleotide sequence has at least 80% identity to SEQ ID NO: 2.

In some embodiments, the third nucleotide sequence has at least 80% identity to SEQ ID NO: 3.

In some embodiments, the determination that SARS-CoV 2 is present in the sample is made also in accordance with a determination that RNAse P gene is present in the sample.

In some embodiments, the determination that SARS-CoV 2 is absent in the sample is made also in accordance with a determination that RNAse P gene is absent in the sample.

In some embodiments, the RNAse P gene is human RNAse P gene.

In some embodiments, the determination that RNAse P gene is present in the sample is made by any method described herein.

In some embodiments, the determination that RNAse P gene is absent in the sample is made by any method described herein.

In some embodiments, the determination that the first nucleotide sequence is present in the sample and the determination that the second nucleotide sequence is present in the sample are made by any method described herein.

In some embodiments, the determination that the first nucleotide sequence is absent in the sample and the determination that the second nucleotide sequence is absent in the sample are made by any method described herein.

In accordance with some embodiments, a reagent kit includes a first amplification primer pair for determining presence of a first nucleotide sequence in a pooled batch of samples; and a second amplification primer pair for determining presence of a second nucleotide sequence in the pooled batch of samples. The reagent kit does not include an amplification primer pair for determining presence of a third nucleotide sequence in the pooled batch of samples, the third nucleotide sequence having at least 80% identity to SEQ ID NO: 3.

In some embodiments, the first nucleotide sequence has at least 80% identity to SEQ ID NO: 1.

In some embodiments, the second nucleotide sequence has at least 80% identity to SEQ ID NO: 2.

In some embodiments, the first amplification primer pair includes an amplification primer having a nucleotide sequence having at least 80% identity to SEQ ID NO: 4.

In some embodiments, the first amplification primer pair includes an amplification primer having a nucleotide sequence having at least 80% identity to SEQ ID NO: 5.

In some embodiments, the second amplification primer pair includes an amplification primer having a nucleotide sequence having at least 80% identity to SEQ ID NO: 6.

In some embodiments, the second amplification primer pair includes an amplification primer having a nucleotide sequence having at least 80% identity to SEQ ID NO: 7.

In some embodiments, the reagent kit includes a first probe having a nucleotide sequence having at least 80% identity to SEQ ID NO: 8.

In some embodiments, the reagent kit includes a second probe having a nucleotide sequence having at least 80% identity to SEQ ID NO: 9.

In some embodiments, the first probe and the second probe have distinct labels.

In some embodiments, the reagent kit includes a third amplification primer pair for detecting presence of RNAse P gene in the pooled batch of samples.

In some embodiments, the third amplification primer pair includes an amplification primer having a nucleotide sequence having at least 80% identity to SEQ ID NO: 10.

In some embodiments, the third amplification primer pair includes an amplification primer having a nucleotide sequence having at least 80% identity to SEQ ID NO: 11.

In some embodiments, the reagent kit further includes a third probe having a nucleotide sequence having at least 80% identity to SEQ ID NO: 12.

In some embodiments, the first probe, the second probe, and the third probe have distinct labels.

In some embodiments, the reagent kit does not include any probe for determining presence of the third nucleotide sequence in the pooled batch of samples.

In accordance with some embodiments, any reagent kit described herein is used for detecting severe acute respiratory syndrome coronavirus 2 (SARS-CoV 2) in a pooled batch of samples.

In accordance with some embodiments, a reagent kit includes a first amplification primer pair for determining presence of a first nucleotide sequence in a sample; and a second amplification primer pair for determining presence of a second nucleotide sequence in the sample. The reagent kit does not include an amplification primer pair for determining presence of a third nucleotide sequence in the sample, the third nucleotide sequence having at least 80% identity to SEQ ID NO: 3.

In some embodiments, the reagent kit includes a third amplification primer pair for detecting presence of RNAse P gene in the sample.

In some embodiments, the reagent kit does not include any probe for determining presence of the third nucleotide sequence in the sample.

In accordance with some embodiments, any reagent kit described herein is used for detecting severe acute respiratory syndrome coronavirus 2 (SARS-CoV 2) in a sample from a subject.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 is a flow diagram illustrating a method of detecting severe acute respiratory syndrome coronavirus 2 in a sample from a subject in accordance with some embodiments.

FIG. 2 is a flow diagram illustrating a method for multiplex detection of severe acute respiratory syndrome coronavirus 2 in a sample from a subject in accordance with some embodiments.

FIG. 3 is a flow diagram illustrating a method for high-throughput detection of severe acute respiratory syndrome coronavirus 2 in a sample from a subject by using a pooled batch of samples in accordance with some embodiments.

FIG. 4 is an example amplification curve for a sample containing a probe for a target on the N1 region of severe acute respiratory syndrome coronavirus 2 and a positive control.

FIG. 5 is an example amplification curve for a sample containing a probe for a target on the N2 region of severe acute respiratory syndrome coronavirus 2 and a positive control.

FIG. 6 is an example amplification curve for a sample containing a probe for a target on the N3 region of severe acute respiratory syndrome coronavirus 2 and a positive control.

FIG. 7 is an example amplification curve for a sample containing a probe for a target on the RP gene and a positive control.

### DETAILED DESCRIPTION

Reference will be made to embodiments, examples of which are illustrated in the accompanying drawings. In the following description, numerous specific details are set forth in order to provide a thorough understanding of the various described embodiments. However, it will be apparent to one of ordinary skill in the art that the various described embodiments may be practiced without these particular details. In other instances, methods, procedures, components, circuits, and networks that are well-known to those of ordinary skill in the art are not described in detail so as not to unnecessarily obscure aspects of the embodiments.

It will be understood that, although the terms first, second, etc. may be used herein to describe various elements, these elements should not be limited by these terms. These terms are only used to distinguish one element from another. For example, a first probe could be termed a second probe, and, similarly, a second probe could be termed a first probe, without departing from the scope of the various described embodiments. The first probe and the second probe are both probes, but they are not the same probe.

The terminology used in the description of the embodiments herein is for the purpose of describing particular embodiments only and is not intended to be limiting of the scope of claims. As used herein, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure. It will also be understood that the term “and/or” as used herein refers to and encompasses any and all possible combinations of one or more of the associated listed items (e.g., “A and/or B” encompasses both A and B, A without B, and B without A). It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “primer” and variants thereof refers to an oligonucleotide that acts as a point of initiation of DNA synthesis in a polymerase chain reaction (PCR). A primer is usually about 15 to about 35 nucleotides in length and hybridizes to a region complementary to the target sequence.

The term “probe” and variants thereof (e.g., detection probe) refers to an oligonucleotide that hybridizes to a target nucleic acid in a PCR reaction. A target sequence refers to a region of nucleic acid that is to be analyzed and comprises the polymorphic site of interest.

Unless defined otherwise, all technical and scientific terms used herein have the same meaning as commonly understood by those of ordinary skill in the art to which the invention pertains. Although any methods and materials similar or equivalent to those described herein can be used in the practice or testing of the present invention, various embodiments of methods and materials are specifically described herein.

As explained above, the CDC test panel announced on Jan. 24, 2020 is prone to high false negatives results in detecting severe acute respiratory syndrome coronavirus 2 (SARS-CoV 2). The CDC panel looks for three markers (labeled N1, N2, and N3). According to the CDC protocol, a specimen (e.g., a sample) is considered positive for SARS-CoV 2 (also called 2019-nCoV) if all markers (N1, N2, N3) cycle threshold growth curve across the threshold line. However, the detection of the N2 marker has been proven particularly unreliable. Thus, at least in part by eliminating the need for testing for the N2 marker, the reagents and methods described herein have fewer false negative results compared to the CDC test panel.

Sequence Listing

| SEQ ID | | |
|---|---|---|
| NO | Description | Oligonucleotide Sequence (5′>3′) |
| 1 | First Target | (ccct)cagatt caactggcag taaccaga(at) |
| | Sequence | |
| 2 | Second Target | (aaga)tcacat tggcacccgc aatcctg(cta) |
| | Sequence | |
| 3 | Third Target | (aaattgc)aca atttgccccc agcgcttcag |
| | Sequence | |
| 4 | First Forward Primer | 5’-GAC CCC AAA ATC AGC GAA AT-3’ |
| 5 | First Reverse Primer | 5’-TCT GGT TAC TGC CAG TTG AAT CTG-3’ |
| 6 | Second Forward | 5’-GGG AGC CTT GAA TAC ACC AAA A-3’ |
| | Primer | |
| 7 | Second Reverse | 5’-TGT AGC ACG ATT GCA GCA TTG-3’ |
| | Primer | |
| 8 | First Probe | 5’-(FAM)-ACC CCG CAT TAC GTT TGG TGG ACC- |
| | | (BHQ-1)-3’ |
| 9 | Second Probe | 5’-(FAM)-AYC AC A TTG GCA CCC GCA ATC |
| | | CTG-(BHQ-1)-3’ |
| 10 | Third Forward | 5’-AGA TTT GGA CCT GCG AGC G-3’ |
| | Primer | |
| 11 | Third Reverse Primer | 5’-GAG CGG CTG TCT CCA CAA GT-3’ |
| 12 | Third Probe | 5’-(FAM)- TTC TGA CCT GAA GGC TCT GCG CG- |
| | | (BHQ)-1-3’ |

In some embodiments, SEQ ID NO 1 is ccctcagatt caactggcag taaccagaat. In some embodiments, SEQ ID NO 1 is cagatt caactggcag taaccagaat. In some embodiments, SEQ ID NO 1 is ccctcagatt caactggcag taaccaga. In some embodiments, SEQ ID NO 1 is cagatt caactggcag taaccaga.

In some embodiments, SEQ ID NO 2 is aagatcacat tggcacccgc aatcctgcta. In some embodiments, SEQ ID NO 2 is tcacat tggcacccgc aatcctgcta. In some embodiments, SEQ ID NO 2 is aagatcacat tggcacccgc aatcctg. In some embodiments, SEQ ID NO 2 is tcacat tggcacccgc aatcctg.

In some embodiments, SEQ ID NO 3 is aaattgcaca atttgccccc agcgcttcag. In some embodiments, SEQ ID NO 3 is aca atttgccccc agcgcttcag.

Although the probes shown above have FAM dye (paired with Black Hole Quencher), other labels and quenchers may be used.

In some embodiments, any two of the first probe, the second probe, and the third probe have distinct labels (although two of the first probe, the second probe, and the third probe may have same labels). In some embodiments, the first probe, the second probe, and the third probe have distinct labels (e.g., each of the first probe, the second probe, and the third probe has a unique label). For example, each of the first probe, the second probe, and the third probe may include a unique label selected from a group including (or consisting of) FAM, TET, JOE, VIC, HEX, NED, ROX, TAMRA, Cy 3, Cy 3.5, Cy 5, Cy 5.5, Biosearch Blue, ATTO dye (e.g., ATTO 390, ATTO 425, ATTO 465, ATTO 488, ATTO 495, ATTO 514, ATTO 520, ATTO 532, ATTO 542, ATTO 550, ATTO 565, ATTO 590, ATTO 594, ATTO 610, ATTO 620, ATTO 633, ATTO 643, etc.), CAL Fluor dye (e.g., CAL Fluor Gold 540, CAL Fluor Orange 560, CAL Fluor Red 590, CAL Fluor Red 610, CAL Fluor Red 635, etc.), Quasar dye (e.g., Quasar 570, Quasar 670, Quasar 705, etc.), Pulsar dye (e.g., Pulsar 650, etc.), ALEXA Fluor 594, LC Red 640, and Texas Red. For example, the first probe may include TET, the second probe may include JOE, and the third probe may include ROX. In another example, the first probe may include FAM, the second probe may include ATTO532, and the third probe may include ATTO550. In yet another example, the first probe may include FAM, the second probe may include VIC, and the third probe may include NED.

In some embodiments, the first probe, the second probe, and the third probe may include any quencher. For example, each of the first probe, the second probe, and the third probe includes any Black Hole Quencher dye (e.g., each of the first probe, the second probe, and the third probe may include any one of: BHQ-0, BHQ-1, BHQ-2, BHQ-3, or BHQ-10). In another example, each of the first probe, the second probe, and the third probe includes a minor groove binder with a nonfluorescent quencher (NFQ).

The use of probes with different labels allows multiplex detection of the target sequences. Instead of performing amplification and detection in separate containers (e.g., wells or tubes) for different target sequences, the amplification and detection of the target sequences may be performed in a single container. This allows reduction of reagents required for running a test per sample, and increases the throughput of tests by allowing more samples to be tested on a single sample plate. In addition, less sample volume is required, as the need for separating the sample into multiple containers is reduced or eliminated. Furthermore, elimination of the separation step simplifies the operational steps, thereby improving the speed of the test and also improving the safety of the operator.

RNA extraction

In accordance with some embodiments, RNA is extracted pursuant to the following procedures.

Equipment and Reagents
14.3 M β-mercaptoethanol (β-ME)
Ethanol (70% and 80%)
Sterile, RNase-free pipet tips
Microcentrifuge
Vortexer
QIAshredder spin column, gDNA Eliminator spin column & RNeasy MinElute spin column
Buffer RLT Plus, Buffer RW1, Buffer RPE & RNase-free water

Protocol: Purification of Total RNA from Animal and Human Cells

1. Sample preparation
a) Prepare and label one set of 1.5 mL microcentrifuge tubes.
b) Centrifuge RNA Collection samples briefly (quick spin down).
c) Mix sample by pipetting sample solution up and down within the RNA Collection vial.
d) Transfer 350 μL RNA sample to the microcentrifuge tubes.
e) centrifuge at 300×g for 5 min
2. Disrupt the cells by adding Buffer RLT Plus
a) β-mercaptoethanol ((3-ME) to Buffer RLT Plus before use. Add 10 μl β-ME per 1 ml Buffer RLT Plus. Dispense in a fume hood and wear appropriate protective clothing. Buffer RLT Plus containing β-ME can be stored at room temperature (15-25° C.) for up to 1 month.
b) For pelleted cells, loosen the cell pellet thoroughly by flicking the tube.

### c) Add 350 μl Buffer RLT Plus.

### d) Vortex to mix

3. Homogenize the lysate
a) Pipet the lysate directly into a QIAshredder spin column (not supplied & (label sample name or number) placed in a 2 ml collection tube, and centrifuge for 2 min at full speed.
4. Transfer the homogenized lysate to a gDNA Eliminator spin column placed in a 2 ml collection tube (supplied). Centrifuge for 30 s at 8000×g (10,000 rpm). Discard the column, and save the flowthrough.
5. Add 350 μl of 70% ethanol to the flow-through from step 4, and mix well by pipetting. Do not centrifuge. Proceed immediately to step 6.
6. Transfer the sample, including any precipitate that may have formed, to an RNeasy MinElute spin column(label sample name or number) placed in a 2 ml collection tube (supplied). Close the lid gently, and centrifuge for 15 sat 8000×g (10,000 rpm). Discard the flow-through.
7. Add 700 μl Buffer RW1 to the RNeasy MinElute spin column. Close the lid gently, and centrifuge for 15 sat 8000×g (10,000 rpm) to wash the spin column membrane. Discard the flow-through.
8. Add 500 μl Buffer RPE to the RNeasy MinElute spin column. Close the lid gently, and centrifuge for 15 sat 8000×g (10,000 rpm) to wash the spin column membrane. Discard the flow-through.
9. Add 500 μl of 80% ethanol to the RNeasy MinElute spin column. Close the lid gently, and centrifuge for 2 min at 8000×g (10,000 rpm) to wash the spin column membrane. Discard the collection tube with the flow-through.
10. Place the RNeasy MinElute spin column in a new 2 ml collection tube (supplied & label sample name or number). Open the lid of the spin column, and centrifuge at full speed for 5 min. Discard the collection tube with the flow-through.
11. Place the RNeasy MinElute spin column in a new 1.5 ml collection tube (supplied & label sample name or number). Add 20 μl RNase-free water directly to the center of the spin column membrane. Close the lid gently, and centrifuge for 1 min at full speed to elute the RNA.

Real-Time Reverse Transcriptase-PCR Panel for Detection of SARS-CoV 2

| | | CATALOG | STORAGE |
|---|---|---|---|
| PCR REAGENT | SUPPLIER | NUMBER | CONDITION |
| TaqPath ™ 1-Step | Life | A15299 | −15° C. to −25° C. |
| RTqPCR Master | Technologies | | |
| Mix, CG | | | |
| Water, UltraPure | Invitrogen | 10977-023 | −15° C. to 30° C. |
| Distilled Water, | | | |
| Protease Free or | | | |
| equivalent | | | |
| T1 Forward-Prier | Avellino | NA | −15° C. to −25° C. |
| T1 Reverse-Primer | Avellino | NA | −15° C. to −25° C. |
| T1 Probe | Avellino | NA | −15° C. to −25° C. |
| T2 Forward-Primer | Avellino | NA | −15° C. to −25° C. |
| T2 Reverse-Primer | Avellino | NA | −15° C. to −25° C. |
| T2 Probe | Avellino | NA | −15° C. to −25° C. |
| RP Forward-Primer | Avellino | NA | −15° C. to −25° C. |
| RP Reverse-Primer | Avellino | NA | −15° C. to −25° C. |
| RP Probe | Avellino | NA | −15° C. to −25° C. |
| T1 Positive Control | Avellino | NA | −15° C. to −25° C. |
| T2 Positive Control | Avellino | NA | −15° C. to −25° C. |
| HSC (Taqman Control | Life tech | 4312660 | −15° C. to −25° C. |
| Genomic DNA-Human, | | | |
| human specimen | | | |
| extraction control) | | | |

| EQUIPMENT | SUPPLIER |
|---|---|
| 7500 Fast Real-Time PCR System | Life Technologies |
| PCR Plate Spinner or equivalent | VWR |

| GENERAL MATERIAL and | | CATALOG |
|---|---|---|
| EQUIPMENT | SUPPLIER | NUMBER |
| MicroAmp ™ Optical 96-Well | Life Technologies | 4346906 |
| Reaction Plate with Barcode | | |
| Splash-free Support Base | Life Technologies | 4312063 |
| MicroAmp ™ Adhesive Film | Life Technologies | 4333183 |
| Applicator or equivalent | | |
| MicroAmp ™ Optical Adhesive | Lite Technologies | 4311971 |
| Film or equivalent | | |
| Microcentrifuge or equivalent | N/A | N/A |
| Vortexer (Capsule) or equivalent | N/A | N/A |
| Single Channel Pipettes | N/A | LTS 1000 μL |
| | | LTS 200 μL |
| | | LTS 20 μL |
| | | LTS 10 μL |
| | | LTS 2 μL |
| Multi-Channel Pipettes | N/A | LA6-1200XLS |
| | | LTS 1200 μL |
| | | LTS 200 μL |
| | | LTS 10 μL |
| Single Channel Repeater Pipettes | N/A | Biohit Epet |
| | | BE-100 |
| Pipette tips, aerosol resistant or | N/A | 21-402-550 |
| equivalent | | 21-402-561 |
| | | 21-402-571 |
| | | 21-402-581 |
| | N/A | 02-707-431 |

In addition, the surface decontaminants, such as RNAse Away™ (Fisher Scientific; cat. #21-236-21) and/or 10% bleach (1:10 dilution of commercial 5.25-6.0% sodium hypochlorite), may be used.

In some cases, the real-time PCT system is configured for Detector (FAM); Quencher (None); Passive Reference: (None); Run Mode: (Standard); Sample Volume (20 μL).

| Step | Cycles | Temp | Time | |
|---|---|---|---|---|
| UNG incubation | 1 | 25° C. | 2 | min |
| RT incubation | 1 | 50° C. | 15 | min |
| Enzyme activation | 1 | 95° C. | 2 | min |
| Amplification | 45 | 95° C. | 3 | sec |
| | | 55° C. | 30 | sec |

### Reaction Master Mix and Plate Set-Up

1) In the reagent set-up room clean hood, place rRT-PCR buffer, enzyme, and primer/probes on ice or cold-block. Keep cold during preparation and use.
2) Thaw TaqPath™ 1-Step RT-qPCR Master Mix prior to use.
3) Mix buffer, enzyme, and primer/probes by inversion 5 times.
4) Briefly centrifuge buffer and primers/probes and return to ice.
5) Label one 1.5 mL microcentrifuge tube.
6) Calculate the amount of each reagent to be added for each reaction mixture including all three primer/probe set (N=# of reactions).

| | | Vol. of Reagent Added per | |
|---|---|---|---|
| Step # | Reagent | Reaction | |
| 1 | Nuclease-free Water | N × 8.5 | μL |
| 2 | Forward primer | N × 0.5 | μL |
| 3 | Reverse primer | N × 0.5 | μL |
| 4 | Probe | N × 0.5 | μL |
| 5 | TaqPath ™ 1-Step RT-qPCR | N × 5 | μL |
| | Master Mix | | |
| | Total Volume | 15 | μL |

7) Dispense reagents into each respective labeled 1.5 mL microcentrifuge tube. After addition of the reagents, mix reaction mixtures by pipetting up and down. Do not vortex.
8) Centrifuge for 5 seconds to collect contents at the bottom of the tube, and then place the tube in a cold rack.
9) Set up reaction strip tubes or plates in a 96-well cooler rack.
10) Dispense 15 μL of each master mix into the appropriate wells (e.g., going across the row).
11) Prior to moving to the nucleic acid handling area, prepare the No Template Control (NTC) reactions for column #1 and C12 well in the assay preparation area.
12) Pipette 5 μL of nuclease-free water into the NTC sample wells.
13) Cover the entire reaction plate and move the reaction plate to the specimen nucleic acid handling area.

### RNA Sample & Control Addition

1) Gently vortex RNA sample tubes for approximately 5 seconds.
2) After centrifugation, place extracted RNA sample tubes in the cold rack.
3) RNA Samples should be added to column 2-10 (column 1, 11 and 12 are for controls). Carefully pipette 5.0 μL of the first sample into a well for that sample (i.e. Sample “Sample 1” in column #2). Change tips after each addition.
4) Add 5 μL of Human Specimen Control (HSC) extracted sample to the HSC wells (column 11).
5) Pipette 5 μL of T1 PTC & T2 PTC to the sample wells of column 12.
6) Securely Seal the plate.
7) Briefly centrifuge reaction plate for 10-15 seconds. After centrifugation put to 7500.
8) Start 7500 rt-PCR.

### Data Analysis

NTCs should be negative and not exhibit fluorescence growth curves that cross the threshold line. If a false positive occurs with one or more of the primer and probe NTC reactions, sample contamination may have occurred. Invalidate the run and repeat the assay with stricter adherence to the procedure guidelines.

Positive Template Control (PTC) reaction should produce a positive result with an expected Ct value for each target included in the test. If expected positive reactivity is not achieved, invalidate the run and repeat the assay with stricter adherence to procedure guidelines. Determine the cause of failed PTC reactivity, implement corrective actions, and document results of the investigation and corrective actions. Do not use PTC reagents that do not generate expected result.

RNAse P (RP) should be positive at or before 35 cycles for all clinical samples and HSC, thus indicating the presence of sufficient nucleic acid from human RNase P gene and that the specimen is of acceptable quality. Failure to detect RNase P in HSC may indicate: 1) improper assay set up and execution and/or 2) reagent or equipment malfunction. Detection of RNase P in human specimen extraction control (HSC) but failure to detect RNase P in any of the clinical samples may indicate: 1) Improper extraction of nucleic acid from clinical materials resulting in loss of nucleic acid or carry-over of PCR inhibitors from clinical specimens and/or 2) absence of sufficient human cellular material in sample to enable detection.

HSC should be negative for 2019-nCoV specific primer/probe sets. If any 2019-nCoV specific primer/probes exhibit a growth curve that crosses the threshold line, interpret as follows: 1) contamination of nucleic acid extraction reagents may have occurred, in which case an operator should invalidate the run and confirm reagent integrity of nucleic acid extraction reagents prior to further testing, or 2) cross contamination of samples occurred during nucleic acid extraction procedures or assay setup, in which case the operator should invalidate the run and repeat the assay with stricter adherence to procedure guidelines.

COVID-19 rRT-PCR Diagnostic Panel Results Interpretation

| T1 Target | T2 Target | RP | Result Interpretation |
|---|---|---|---|
| + | + | ± | 2019-nCoV detected |
| | | | Positive Result |
| If only one, or two targets is positive | | ± | Inconclusive Result |
| − | − | + | 2019-nCoV not detected |
| | | | Negative Result |
| − | − | − | Invalid Result |

Although the methods and reagents have been described with respect to the primers and probes described above, other primers and probes may be used.

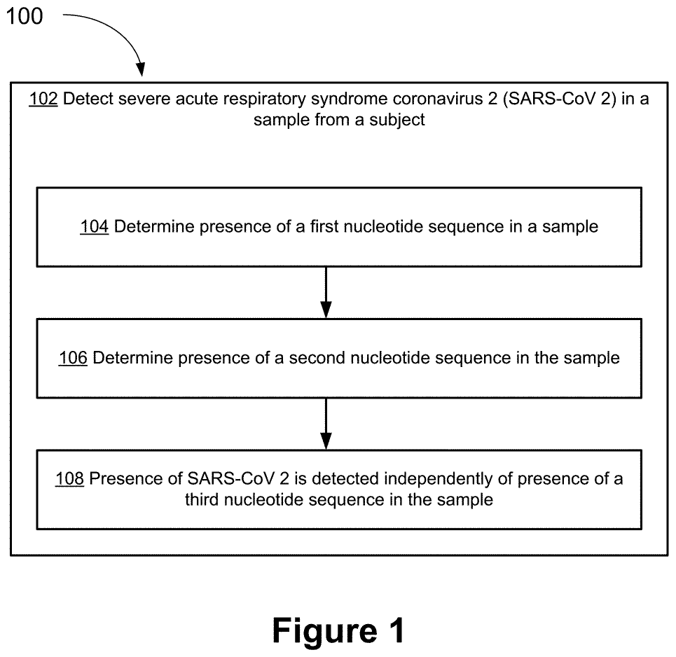

FIG. 1 is a flow diagram illustrating a method 100 of detecting severe acute respiratory syndrome coronavirus 2 in a sample from a subject in accordance with some embodiments.

The method 100 includes (102) detecting severe acute respiratory syndrome coronavirus 2 in a sample from a subject by (104) determining presence of a first nucleotide sequence (e.g., corresponding to the N1 region) in a sample and (106) determining presence of a second nucleotide sequence (e.g., corresponding to the N3 region) in the sample. Presence of SARS-CoV 2 is detected (108) independently of determining presence of a third nucleotide sequence (e.g., corresponding to the N2 region) in the sample (e.g., without detecting the presence of the third nucleotide sequence in the sample or disregarding the presence of the third nucleotide sequence in the sample).

In some embodiments, the presence of the first nucleotide sequence in the sample is determined prior to determining the presence of the second nucleotide sequence in the sample. In some embodiments, the presence of the first nucleotide sequence in the sample is determined after determining the presence of the second nucleotide sequence in the sample. In some embodiments, the presence of the first nucleotide sequence in the sample is determined concurrently with determining the presence of the second nucleotide sequence in the sample.

In some embodiments, the presence of the first nucleotide sequence in the sample is determined using a first amplification primer primer pair. In some embodiments, the presence of the second nucleotide sequence in the sample is determined using a second amplification primer primer pair.

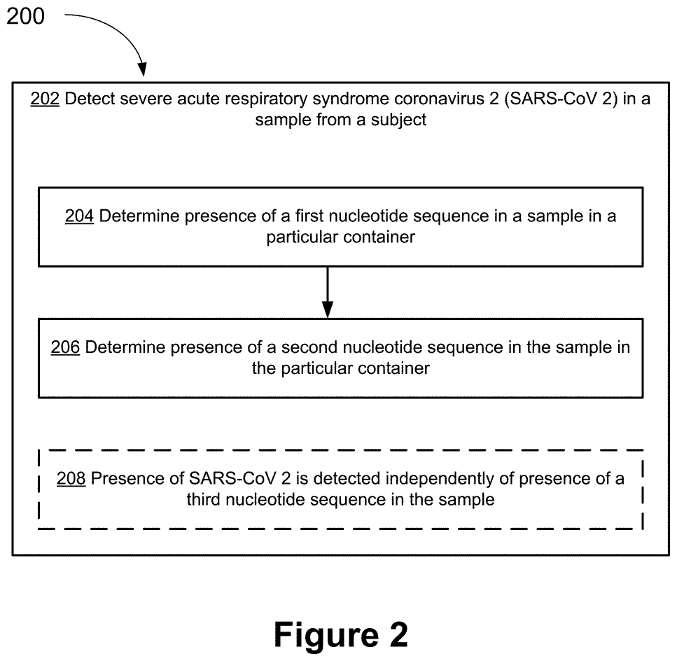

FIG. 2 is a flow diagram illustrating a method 200 for multiplex detection of severe acute respiratory syndrome coronavirus 2 in a sample from a subject in accordance with some embodiments.

The method 200 includes (202) detecting severe acute respiratory syndrome coronavirus 2 (SARS-CoV 2) in a sample from a subject by (204) determining presence of a first nucleotide sequence in a sample in a particular container and (206) determining presence of a second nucleotide sequence in the sample in the particular container. In some embodiments, presence of SARS-CoV 2 is detected (208) independently of determining presence of a third nucleotide sequence (e.g., corresponding to the N2 region) in the sample (e.g., without detecting the presence of the third nucleotide sequence in the sample or disregarding the presence of the third nucleotide sequence in the sample).

In some embodiments, the presence of the first nucleotide sequence in the sample is determined prior to determining the presence of the second nucleotide sequence in the sample. In some embodiments, the presence of the first nucleotide sequence in the sample is determined after determining the presence of the second nucleotide sequence in the sample. In some embodiments, the presence of the first nucleotide sequence in the sample is determined concurrently with determining the presence of the second nucleotide sequence in the sample.

In some embodiments, the presence of the first nucleotide sequence in the sample is determined using a first amplification primer primer pair. In some embodiments, the presence of the second nucleotide sequence in the sample is determined using a second amplification primer primer pair.

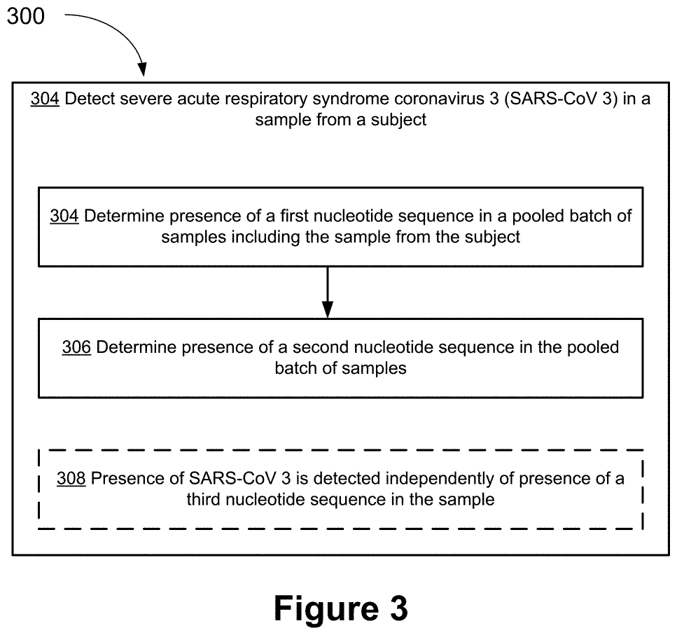

FIG. 3 is a flow diagram illustrating a method 300 for high-throughput detection of severe acute respiratory syndrome coronavirus 2 in a sample from a subject by using a pooled batch of samples in accordance with some embodiments.

The method 300 includes (302) detecting severe acute respiratory syndrome coronavirus 2 (SARS-CoV 2) in a sample from a subject by (304) determining presence of a first nucleotide sequence in a pooled batch of samples including the sample from the subject and (306) determining presence of a second nucleotide sequence in the pooled batch of samples. In some embodiments, presence of SARS-CoV 2 is detected (308) independently of determining presence of a third nucleotide sequence (e.g., corresponding to the N2 region) in the sample (e.g., without detecting the presence of the third nucleotide sequence in the sample or disregarding the presence of the third nucleotide sequence in the sample).

In some embodiments, the presence of the first nucleotide sequence in the sample is determined prior to determining the presence of the second nucleotide sequence in the sample. In some embodiments, the presence of the first nucleotide sequence in the sample is determined after determining the presence of the second nucleotide sequence in the sample. In some embodiments, the presence of the first nucleotide sequence in the sample is determined concurrently with determining the presence of the second nucleotide sequence in the sample.

In some embodiments, the presence of the first nucleotide sequence in the sample is determined using a first amplification primer primer pair. In some embodiments, the presence of the second nucleotide sequence in the sample is determined using a second amplification primer primer pair.

### Examples

Detection of SARS-CoV2 with and without N2 Target

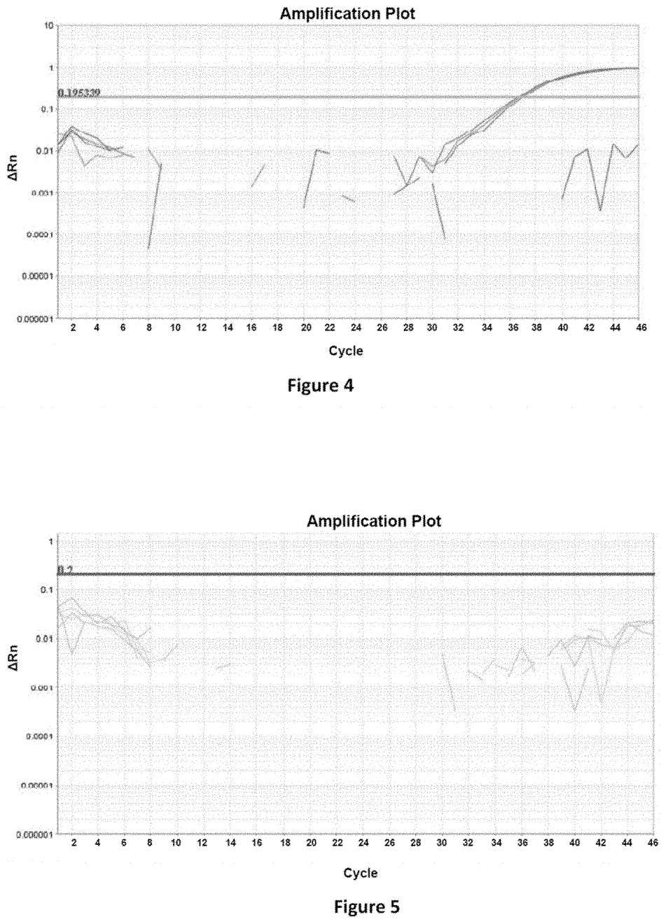

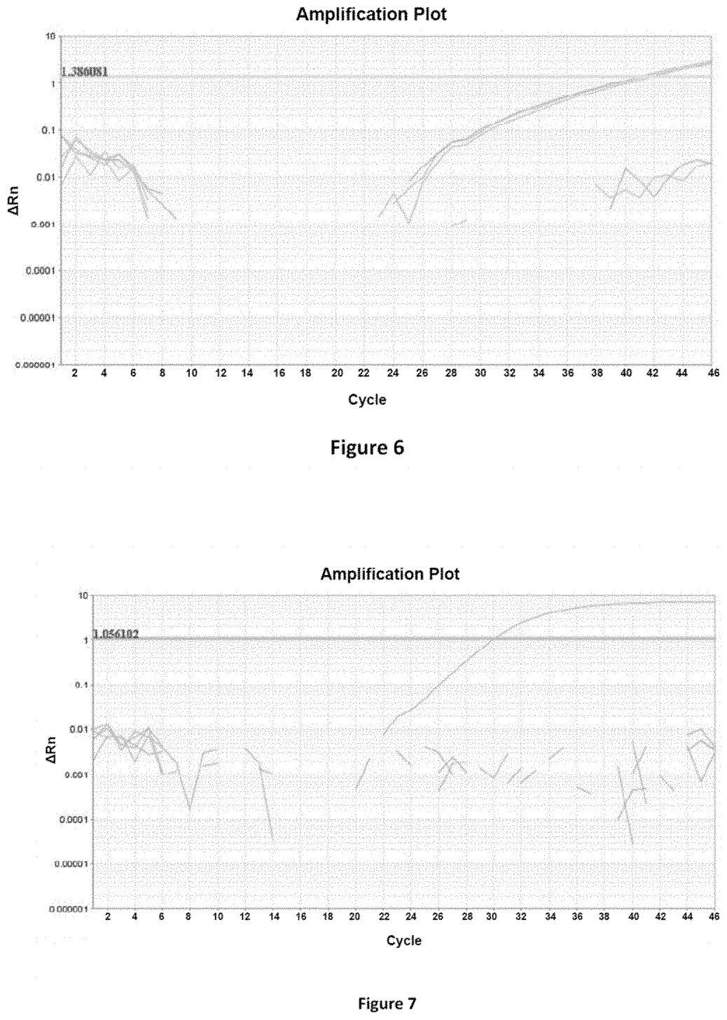

FIG. 4 is an example amplification curve for a sample containing a probe for a target on the N1 region of severe acute respiratory syndrome coronavirus 2 and a positive control. FIG. 5 is an example amplification curve for a sample containing a probe for a target on the N2 region of severe acute respiratory syndrome coronavirus 2 and a positive control. FIG. 6 is an example amplification curve for a sample containing a probe for a target on the N3 region of severe acute respiratory syndrome coronavirus 2 and a positive control. FIG. 7 is an example amplification curve for a sample containing a probe for a target on the RP gene and a positive control. As shown in FIGS. 4-7, the N1 region, the N3 region, and the RP gene are detected from the positive control, whereas the N2 region is not detected.

### High-Throughput Detection by Pooling

To demonstrate the validity of pooling, various samples were spiked with differnt numbers of positive samples. Sample volume is 100 uL per sample. The positive-versus-negative-sample ratios are as follows:

a. 5 positives:5 negatives

b. 4 positives:6 negatives

c. 3 positives:7 negatives

d. 2 positives:8 negatives

e. 1 positive:9 negatives

f 1 positive:19 negatives

g. 1 positive:39 negatives

h. 1 positive:49 negatives

i. 1 positive:59 negatives

j. 1 positive:69 negatives

k. 1 positive:79 negatives

l. 1 positive:89 negatives

m. 1 positive:99 negatives

n. 1 positive:109 negatives

o. 1 positive:119 negatives

These pooled samples were analyzed using the multiplex rRT-PCR method described herein.

a. Protocol

1. Making sample pool: Randomly select 120 known negative patient samples and make a sample pool by pipetting 100 uL from each sample. Randomly select one positive sample that has a Ct value around 15. Pipette 100 uL of the positive sample into the negative samples according to the flowing scheme. Mx well and make three sets of triplicates.
Sample 1: 1 positive:9 negatives
Sample 2: 2 positives:8 negatives
Sample 3: 3 positives:7 negatives
Sample 4: 4 positives:6 negatives
Sample 5: 5 positives:5 negatives
Sample 6: 1 positive:19 negatives
Sample 7: 1 positive:39 negatives
Sample 8: 1 positive:49 negatives
Sample 9: 1 positive:59 negatives
Sample 10: 1 positive:69 negatives
Sample 11: 1 positive:79 negatives
Sample 12: 1 positive:89 negatives
Sample 13: 1 positive:99 negatives
Sample 14: 1 positive:109 negatives
Sample 15: 1 positive:119 negatives

2. Using QIAcube for automated RNA extraction in 96-well format: Set up the QIAcube with the three sets of triplicates on the same sample deep well plate and proceed using the instrument to perform automated RNA extraction.

3. Perform rRT-PCR with the multiplex method described herein.

b. Results

Results for samples tested using the Avellino SARS-CoV-2 Reverse Transcriptase RT-PCR test are shown in the table below.

| | # of | # of | | | | |
|---|---|---|---|---|---|---|
| Sample | Pos- | Neg- | Dilution | T1 Ct | T2 Ct | RP Ct |
| # | itive | ative | factor | value | value | value |
| 1-1 | 1 | 9 | 1:10 | 24.75 | 24.07 | 25.50 |
| 1-2 | | | | 25.57 | 24.56 | 25.98 |
| 1-3 | | | | 25.16 | 24.55 | 25.98 |
| 2-1 | 2 | 8 | 2:10 | 24.86 | 24.27 | 25.92 |
| 2-2 | | | | 24.95 | 24.32 | 25.84 |
| 2-3 | | | | 25.47 | 24.78 | 26.34 |
| 3-1 | 3 | 7 | 3:10 | 24.95 | 24.29 | 25.84 |
| 3-2 | | | | 23.65 | 23.02 | 24.68 |
| 3-3 | | | | 26.97 | 24.71 | 26.35 |
| 4-1 | 4 | 6 | 4:10 | 19.74 | 19.37 | 21.18 |
| 4-2 | | | | 20.38 | 20.06 | 21.79 |
| 4-3 | | | | 21.76 | 21.22 | 23.05 |
| 5-1 | 5 | 5 | 5:10 | 22.16 | 21.75 | 23.62 |
| 5-2 | | | | 22.10 | 21.72 | 23.58 |
| 5-3 | | | | 22.87 | 22.18 | 23.97 |
| 6-1 | 1 | 19 | 1:20 | 29.33 | 27.23 | 27.74 |
| 6-2 | | | | 28.87 | 26.87 | 27.44 |
| 6-3 | | | | 28.47 | 26.62 | 27.17 |
| 7-1 | 1 | 39 | 1:40 | 29.40 | 27.17 | 27.66 |
| 7-2 | | | | 29.42 | 27.27 | 27.69 |
| 7-3 | | | | 29.36 | 27.24 | 27.58 |
| 8-1 | 1 | 49 | 1:50 | 30.32 | 26.82 | 27.44 |
| 8-2 | | | | 30.29 | 27.98 | 28.23 |
| 8-3 | | | | 30.09 | 27.50 | 27.77 |
| 9-1 | 1 | 59 | 1:60 | 30.21 | 27.50 | 27.84 |
| 9-2 | | | | 30.12 | 27.39 | 27.64 |
| 9-3 | | | | 30.03 | 27.43 | 27.65 |
| 10-1 | 1 | 69 | 1:70 | 33.20 | 27.84 | 27.86 |
| 10-2 | | | | 33.36 | 27.79 | 27.87 |
| 10-3 | | | | 32.36 | 27.40 | 27.45 |
| 11-1 | 1 | 79 | 1:80 | 32.95 | 27.78 | 27.79 |
| 11-2 | | | | 33.47 | 27.72 | 27.74 |
| 11-3 | | | | 33.34 | 27.39 | 27.46 |
| 12-1 | 1 | 89 | 1:90 | 33.88 | 27.69 | 27.77 |
| 12-2 | | | | 33.44 | 27.64 | 27.62 |
| 12-3 | | | | 33.59 | 27.75 | 27.82 |
| 13-1 | 1 | 99 | 1:100 | 33.81 | 27.90 | 27.83 |
| 13-2 | | | | 25.88 | 24.68 | 25.84 |
| 13-3 | | | | 33.34 | 27.45 | 27.59 |
| 14-1 | 1 | 109 | 1:110 | 33.13 | 27.55 | 27.60 |
| 14-2 | | | | 25.77 | 24.69 | 25.81 |
| 14-3 | | | | 33.61 | 28.04 | 27.93 |
| 15-1 | 1 | 119 | 1:120 | 33.58 | 27.74 | 27.75 |
| 15-2 | | | | 26.95 | 24.73 | 25.76 |
| 15-3 | | | | 34.02 | 28.08 | 27.90 |
| NTC | | | | Undeter- | Undeter- | Undeter- |
| | | | | mined | mined | mined |
| EC | | | | Undeter- | 27.03 | 27.00 |
| | | | | mined | | |
| PTC | | | | 28.58 | 26.79 | 27.65 |

c. Conclusion

The data demonstrates that the one positive sample, mixed with as many as 119 negative samples, can be detected by the method described herein.

Some embodiments are described with respect to the following clauses.

Clause 1. A method for detecting severe acute respiratory syndrome coronavirus 2 (SARS-CoV 2) in a sample from a subject, the method comprising:
determining presence of a first nucleotide sequence in a sample; and
determining presence of a second nucleotide sequence in the sample,
wherein presence of SARS-CoV 2 is detected independently of determining presence of a third nucleotide sequence in the sample.
Clause 2. The method of clause 1, wherein the first nucleotide sequence has at least 80% identity to SEQ ID NO: 1.
Clause 3. The method of clause 1 or 2, wherein the second nucleotide sequence has at least 80% identity to SEQ ID NO: 2.
Clause 4. The method of any of clauses 1-3, wherein the third nucleotide sequence has at least 80% identity to SEQ ID NO: 3.
Clause 5. The method of any of clauses 1-4, wherein the presence of the first nucleotide sequence in the sample is determined using a first amplification primer pair and the first amplification primer pair includes an amplification primer having a nucleotide sequence having at least 80% identity to SEQ ID NO: 4.
Clause 6. The method of any of clauses 1-5, wherein the presence of the first nucleotide sequence in the sample is determined using a first amplification primer pair and the first amplification primer pair includes an amplification primer having a nucleotide sequence having at least 80% identity to SEQ ID NO: 5.
Clause 7. The method of any of clauses 1-6, wherein the presence of the second nucleotide sequence in the sample is determined using a second amplification primer pair and the second amplification primer pair includes an amplification primer having a nucleotide sequence having at least 80% identity to SEQ ID NO: 6.
Clause 8. The method of any of clauses 1-7, wherein the presence of the second nucleotide sequence in the sample is determined using a second amplification primer pair and the second amplification primer pair includes an amplification primer having a nucleotide sequence having at least 80% identity to SEQ ID NO: 7.
Clause 9. The method of any of clauses 1-8, wherein the presence of the first nucleotide sequence is determined also using a first probe having a nucleotide sequence having at least 80% identity to SEQ ID NO: 8.
Clause 10. The method of any of clauses 1-9, wherein the presence of the second nucleotide sequence is determined also using a second probe having a nucleotide sequence having at least 80% identity to SEQ ID NO: 9.
Clause 11. The method of any of clauses 1-10, further comprising:
determining presence of RNAse P gene in the sample using a third amplification primer pair.
Clause 12. The method of clause 11, wherein the third amplification primer pair includes an amplification primer having a nucleotide sequence having at least 80% identity to SEQ ID NO: 10.
Clause 13. The method of clause 11 or 12, wherein the third amplification primer pair includes an amplification primer having a nucleotide sequence having at least 80% identity to SEQ ID NO: 11.
Clause 14. The method of any of clauses 11-13, wherein presence of RNAse P gene in the sample is determined also using a third probe having a nucleotide sequence having at least 80% identity to SEQ ID NO: 12.
Clause 15. The method of any of clauses 11-14, wherein the presence of RNAse P gene is detected by real-time reverse transcription polymerase chain reaction.
Clause 16. The method of any of clauses 11-15, wherein the RNAse P gene is human RNAse P gene.
Clause 17. The method of any of clauses 1-16, wherein the presence of the first nucleotide sequence is detected by real-time reverse transcription polymerase chain reaction.
Clause 18. The method of any of clauses 1-17, wherein the presence of the second nucleotide sequence is detected by real-time reverse transcription polymerase chain reaction.
Clause 19. The method of any of clauses 1-18, wherein the subject is a human.
Clause 20. A method for detecting severe acute respiratory syndrome coronavirus 2 (SARS-CoV 2) in a sample from a subject, the method comprising:
determining that SARS-CoV 2 is present in the sample in accordance with at least a determination that a first nucleotide sequence is present in a sample and a determination that a second nucleotide sequence is present in the sample independent of whether a third nucleotide sequence is present in the sample.
Clause 21. The method of clause 20, wherein the first nucleotide sequence has at least 80% identity to SEQ ID NO: 1.
Clause 22. The method of clause 20 or 21, wherein the second nucleotide sequence has at least 80% identity to SEQ ID NO: 2.
Clause 23. The method of any of clauses 20-22, wherein the third nucleotide sequence has at least 80% identity to SEQ ID NO: 3.
Clause 24. The method of any of clauses 20-23, wherein the determination that SARS-CoV 2 is present in the sample is made also in accordance with a determination that RNAse P gene is present in the sample.
Clause 25. The method of clause 24, wherein the RNAse P gene is human RNAse P gene.
Clause 26. The method of clause 24 or 25, wherein the determination that RNAse P gene is present in the sample is made by the method of any of clauses 11-16.
Clause 27. The method of any of clauses 20-26, wherein the determination that the first nucleotide sequence is present in the sample and the determination that the second nucleotide sequence is present in the sample are made by the method of any of clauses 1-19.
Clause 28. A reagent kit, comprising:
a first amplification primer pair for determining presence of a first nucleotide sequence in a sample; and
a second amplification primer pair for determining presence of a second nucleotide sequence in the sample,
wherein the reagent kit does not include an amplification primer pair for determining presence of a third nucleotide sequence in the sample, the third nucleotide sequence having at least 80% identity to SEQ ID NO: 3.
Clause 29. The reagent kit of clause 28, wherein the first nucleotide sequence has at least 80% identity to SEQ ID NO: 1.
Clause 30. The reagent kit of clause 28 or 29, wherein the second nucleotide sequence has at least 80% identity to SEQ ID NO: 2.
Clause 31. The reagent kit of any of clauses 28-30, wherein the first amplification primer pair includes an amplification primer having a nucleotide sequence having at least 80% identity to SEQ ID NO: 4.
Clause 32. The reagent kit of any of clauses 28-31, wherein the first amplification primer pair includes an amplification primer having a nucleotide sequence having at least 80% identity to SEQ ID NO: 5.
Clause 33. The reagent kit of any of clauses 28-32, wherein the second amplification primer pair includes an amplification primer having a nucleotide sequence having at least 80% identity to SEQ ID NO: 6.
Clause 34. The reagent kit of any of clauses 28-33, wherein the second amplification primer pair includes an amplification primer having a nucleotide sequence having at least 80% identity to SEQ ID NO: 7.
Clause 35. The reagent kit of any of clauses 28-34, further comprising a first probe having a nucleotide sequence having at least 80% identity to SEQ ID NO: 8.
Clause 36. The reagent kit of any of clauses 28-35, further comprising a second probe having a nucleotide sequence having at least 80% identity to SEQ ID NO: 9.
Clause 37. The reagent kit of any of clauses 28-36, further comprising a third amplification primer pair for detecting presence of RNAse P gene in the sample.
Clause 38. The reagent kit of clause 37, wherein the third amplification primer pair includes an amplification primer having a nucleotide sequence having at least 80% identity to SEQ ID NO: 10.
Clause 39. The reagent kit of clause 37 or 38, wherein the third amplification primer pair includes an amplification primer having a nucleotide sequence having at least 80% identity to SEQ ID NO: 11.
Clause 40. The reagent kit of any of clauses 37-39, further comprising a third probe having a nucleotide sequence having at least 80% identity to SEQ ID NO: 12.
Clause 41. The reagent kit of any of clauses 37-40, wherein the reagent kit does not include any probe for determining presence of the third nucleotide sequence in the sample.
Clause 42. Use of the reagent kit of any of clauses 28-41 for detecting severe acute respiratory syndrome coronavirus 2 (SARS-CoV 2) in a sample from a subject.
Clause 43. A method for detecting severe acute respiratory syndrome coronavirus 2 (SARS-CoV 2) in a sample from a subject, the method comprising:
determining presence of a first nucleotide sequence in a sample in a particular container; and determining presence of a second nucleotide sequence in the sample in the particular container.
Clause 44. The method of clause 43, wherein the first nucleotide sequence has at least 80% identity to SEQ ID NO: 1.
Clause 45. The method of clause 43 or 44, wherein the second nucleotide sequence has at least 80% identity to SEQ ID NO: 2.
Clause 46. The method of any of clauses 43-45, wherein presence of SARS-CoV 2 is detected independently of determining presence of a third nucleotide sequence in the sample and the third nucleotide sequence has at least 80% identity to SEQ ID NO: 3.
Clause 47. The method of any of clauses 43-46, wherein the presence of the first nucleotide sequence in the sample is determined using a first amplification primer pair and the first amplification primer pair includes an amplification primer having a nucleotide sequence having at least 80% identity to SEQ ID NO: 4.
Clause 48. The method of any of clauses 43-47, wherein the presence of the first nucleotide sequence in the sample is determined using a first amplification primer pair and the first amplification primer pair includes an amplification primer having a nucleotide sequence having at least 80% identity to SEQ ID NO: 5.
Clause 49. The method of any of clauses 43-48, wherein the presence of the second nucleotide sequence in the sample is determined using a second amplification primer pair and the second amplification primer pair includes an amplification primer having a nucleotide sequence having at least 80% identity to SEQ ID NO: 6.
Clause 50. The method of any of clauses 43-49, wherein the presence of the second nucleotide sequence in the sample is determined using a second amplification primer pair and the second amplification primer pair includes an amplification primer having a nucleotide sequence having at least 80% identity to SEQ ID NO: 7.
Clause 51. The method of any of clauses 43-50, wherein the presence of the first nucleotide sequence is determined also using a first probe having a nucleotide sequence having at least 80% identity to SEQ ID NO: 8.
Clause 52. The method of any of clauses 43-51, wherein the presence of the second nucleotide sequence is determined also using a second probe having a nucleotide sequence having at least 80% identity to SEQ ID NO: 9.
Clause 53. The method of clause 52, wherein the first probe and the second probe have distinct labels.
Clause 54. The method of any of clauses 43-53, further comprising:
determining presence of RNAse P gene in the sample in the particular container using a third amplification primer pair.
Clause 55. The method of clause 54, wherein the third amplification primer pair includes an amplification primer having a nucleotide sequence having at least 80% identity to SEQ ID NO: 10.
Clause 56. The method of clause 54 or 55, wherein the third amplification primer pair includes an amplification primer having a nucleotide sequence having at least 80% identity to SEQ ID NO: 11.
Clause 57. The method of any of clauses 54-56, wherein presence of RNAse P gene in the sample is determined also using a third probe having a nucleotide sequence having at least 80% identity to SEQ ID NO: 54.
Clause 58. The method of clause 57, wherein the first probe, the second probe, and the third probe have distinct labels.
Clause 59. The method of any of clauses 54-58, wherein the presence of RNAse P gene is detected by real-time reverse transcription polymerase chain reaction.
Clause 60. The method of any of clauses 54-59, wherein the RNAse P gene is human RNAse P gene.
Clause 61. The method of any of clauses 43-60, wherein the presence of the first nucleotide sequence is detected by real-time reverse transcription polymerase chain reaction while the sample is in the particular container.
Clause 62. The method of any of clauses 43-61, wherein the presence of the second nucleotide sequence is detected by real-time reverse transcription polymerase chain reaction while the sample is in the particular container.
Clause 63. The method of clause 62, wherein the presence of the first nucleotide sequence and the presence of the second nucleotide sequence are detected by concurrent real-time transcription polymerase chain reaction.
Clause 64. The method of any of clauses 43-63, wherein the subject is a human.
Clause 65. A method for detecting severe acute respiratory syndrome coronavirus 2 (SARS-CoV 2) in a sample from a subject, the method comprising:
determining that SARS-CoV 2 is present in the sample in accordance with at least a determination that a first nucleotide sequence is present in the sample in a particular container and a determination that a second nucleotide sequence is present in the sample in the particular container.
Clause 66. The method of clause 65, wherein the first nucleotide sequence has at least 80% identity to SEQ ID NO: 1.
Clause 67. The method of clause 65 or 66, wherein the second nucleotide sequence has at least 80% identity to SEQ ID NO: 2.
Clause 68. The method of any of clauses 65-67, wherein presence of SARS-CoV 2 is detected independently of determining presence of a third nucleotide sequence in the sample and the third nucleotide sequence has at least 80% identity to SEQ ID NO: 3.
Clause 69. The method of any of clauses 65-68, wherein the determination that SARS-CoV 2 is present in the sample is made also in accordance with a determination that RNAse P gene is present in the sample.
Clause 70. The method of clause 69, wherein the RNAse P gene is human RNAse P gene.
Clause 71. The method of clause 69 or 70, wherein the determination that RNAse P gene is present in the sample is made by the method of any of clauses 54-18.
Clause 72. The method of any of clauses 65-71, wherein the determination that the first nucleotide sequence is present in the sample and the determination that the second nucleotide sequence is present in the sample are made by the method of any of clauses 43-22.
Clause 73. A reagent kit, comprising:
a first amplification primer pair for determining presence of a first nucleotide sequence in a sample; and
a second amplification primer pair for determining presence of a second nucleotide sequence in the sample,
wherein the reagent kit does not include an amplification primer pair for determining presence of a third nucleotide sequence in the sample, the third nucleotide sequence having at least 80% identity to SEQ ID NO: 3.
Clause 74. The reagent kit of clause 73, wherein the first nucleotide sequence has at least 80% identity to SEQ ID NO: 1.
Clause 75. The reagent kit of clause 73 or 74, wherein the second nucleotide sequence has at least 80% identity to SEQ ID NO: 2.
Clause 76. The reagent kit of any of clauses 73-75, wherein the first amplification primer pair includes an amplification primer having a nucleotide sequence having at least 80% identity to SEQ ID NO: 4.
Clause 77. The reagent kit of any of clauses 73-76, wherein the first amplification primer pair includes an amplification primer having a nucleotide sequence having at least 80% identity to SEQ ID NO: 5.
Clause 78. The reagent kit of any of clauses 73-77, wherein the second amplification primer pair includes an amplification primer having a nucleotide sequence having at least 80% identity to SEQ ID NO: 6.
Clause 79. The reagent kit of any of clauses 73-78, wherein the second amplification primer pair includes an amplification primer having a nucleotide sequence having at least 80% identity to SEQ ID NO: 7.
Clause 80. The reagent kit of any of clauses 73-79, further comprising a first probe having a nucleotide sequence having at least 80% identity to SEQ ID NO: 8.
Clause 81. The reagent kit of any of clauses 73-80, further comprising a second probe having a nucleotide sequence having at least 80% identity to SEQ ID NO: 9.
Clause 82. The reagent kit of clause 81, wherein the first probe and the second probe have distinct labels.
Clause 83. The reagent kit of any of clauses 73-82, further comprising a third amplification primer pair for detecting presence of RNAse P gene in the sample.
Clause 84. The reagent kit of clause 83, wherein the third amplification primer pair includes an amplification primer having a nucleotide sequence having at least 80% identity to SEQ ID NO: 10.
Clause 85. The reagent kit of clause 83 or 84, wherein the third amplification primer pair includes an amplification primer having a nucleotide sequence having at least 80% identity to SEQ ID NO: 11.
Clause 86. The reagent kit of any of clauses 83-85, further comprising a third probe having a nucleotide sequence having at least 80% identity to SEQ ID NO: 54.
Clause 87. The reagent kit of clause 86, wherein the first probe, the second probe, and the third probe have distinct labels.
Clause 88. The reagent kit of any of clauses 73-87, wherein the reagent kit does not include any probe for determining presence of the third nucleotide sequence in the sample.
Clause 89. Use of the reagent kit of any of clauses 73-88 for detecting severe acute respiratory syndrome coronavirus 2 (SARS-CoV 2) in a sample from a subject.
Clause 90. A method for detecting severe acute respiratory syndrome coronavirus 2 (SARS-CoV 2) in a sample from a subject, the method comprising:
determining presence of a first nucleotide sequence in a pooled batch of samples including the sample from the subject; and
determining presence of a second nucleotide sequence in the pooled batch of samples.
Clause 91. The method of clause 90, wherein the first nucleotide sequence has at least 80% identity to SEQ ID NO: 1.
Clause 92. The method of clause 90 or 91, wherein the second nucleotide sequence has at least 80% identity to SEQ ID NO: 2.
Clause 93. The method of any of clauses 90-92, wherein presence of SARS-CoV 2 is detected independently of determining presence of a third nucleotide sequence in the sample and the third nucleotide sequence has at least 80% identity to SEQ ID NO: 3.
Clause 94. The method of any of clauses 90-93, wherein the presence of the first nucleotide sequence in the sample is determined using a first amplification primer pair and the first amplification primer pair includes an amplification primer having a nucleotide sequence having at least 80% identity to SEQ ID NO: 4.
Clause 95. The method of any of clauses 90-94, wherein the presence of the first nucleotide sequence in the sample is determined using a first amplification primer pair and the first amplification primer pair includes an amplification primer having a nucleotide sequence having at least 80% identity to SEQ ID NO: 5.
Clause 96. The method of any of clauses 90-95, wherein the presence of the second nucleotide sequence in the sample is determined using a second amplification primer pair and the second amplification primer pair includes an amplification primer having a nucleotide sequence having at least 80% identity to SEQ ID NO: 6.
Clause 97. The method of any of clauses 90-96, wherein the presence of the second nucleotide sequence in the sample is determined using a second amplification primer pair and the second amplification primer pair includes an amplification primer having a nucleotide sequence having at least 80% identity to SEQ ID NO: 7.
Clause 98. The method of any of clauses 90-97, wherein the presence of the first nucleotide sequence is determined also using a first probe having a nucleotide sequence having at least 80% identity to SEQ ID NO: 8.
Clause 99. The method of any of clauses 90-98, wherein the presence of the second nucleotide sequence is determined also using a second probe having a nucleotide sequence having at least 80% identity to SEQ ID NO: 9.
Clause 100. The method of any of clauses 90-10, wherein the first probe and the second probe have distinct labels.
Clause 101. The method of any of clauses 90-100, further comprising:
determining presence of RNAse P gene in the pooled batch of samples using a third amplification primer pair.
Clause 102. The method of clause 101, wherein the third amplification primer pair includes an amplification primer having a nucleotide sequence having at least 80% identity to SEQ ID NO: 10.
Clause 103. The method of clause 101 or 102, wherein the third amplification primer pair includes an amplification primer having a nucleotide sequence having at least 80% identity to SEQ ID NO: 100.
Clause 104. The method of any of clauses 101-103, wherein presence of RNAse P gene in the pooled batch of samples is determined also using a third probe having a nucleotide sequence having at least 80% identity to SEQ ID NO: 101.
Clause 105. The method of clause 104, wherein the first probe, the second probe, and the third probe have distinct labels.
Clause 106. The method of any of clauses 101-105, wherein the presence of RNAse P gene is detected by real-time reverse transcription polymerase chain reaction.
Clause 107. The method of any of clauses 101-106, wherein the RNAse P gene is human RNAse P gene.
Clause 108. The method of any of clauses 90-107, wherein the presence of the first nucleotide sequence is detected by real-time reverse transcription polymerase chain reaction while the pooled batch of samples is in a particular container.
Clause 109. The method of clause 108, wherein the presence of the second nucleotide sequence is detected by real-time reverse transcription polymerase chain reaction while the pooled batch of samples is in the particular container.
Clause 110. The method of clause 109, wherein the presence of the first nucleotide sequence and the presence of the second nucleotide sequence are detected by concurrent real-time transcription polymerase chain reaction.
Clause 111. The method of any of clauses 108-110, wherein the presence of the RNAse P gene is detected by real-time reverse transcription polymerase chain reaction while the pooled batch of samples is in the particular container.
Clause 112. The method of any of clauses 90-111, wherein the subject is a human.
Clause 113. A method for determining absence of severe acute respiratory syndrome coronavirus 2 (SARS-CoV 2) in a sample from a subject, the method comprising: determining that SARS-CoV 2 is absent in the sample in accordance with at least a determination that a first nucleotide sequence is absent in a pooled batch of samples including the sample and a determination that a second nucleotide sequence is absent in the pooled batch of samples.
Clause 114. The method of clause 113, wherein the first nucleotide sequence has at least 80% identity to SEQ ID NO: 1.
Clause 115. The method of clause 113 or 114, wherein the second nucleotide sequence has at least 80% identity to SEQ ID NO: 2.
Clause 116. The method of any of clauses 113-115, wherein presence of SARS-CoV 2 is detected independently of determining presence of a third nucleotide sequence in the sample and the third nucleotide sequence has at least 80% identity to SEQ ID NO: 3.
Clause 117. The method of any of clauses 113-116, wherein the determination that SARS-CoV 2 is absent in the sample is made also in accordance with a determination that RNAse P gene is absent in the sample.
Clause 118. The method of clause 117, wherein the RNAse P gene is human RNAse P gene.
Clause 119. The method of clause 117 or 118, wherein the determination that RNAse P gene is absent in the sample is made by the method of any of clauses 101-107.
Clause 120. The method of any of clauses 113-119, wherein the determination that the first nucleotide sequence is absent in the sample and the determination that the second nucleotide sequence is absent in the sample are made by the method of any of clauses 90-112.
Clause 121. A reagent kit, comprising:
a first amplification primer pair for determining presence of a first nucleotide sequence in a pooled batch of samples; and
a second amplification primer pair for determining presence of a second nucleotide sequence in the pooled batch of samples,
wherein the reagent kit does not include an amplification primer pair for determining presence of a third nucleotide sequence in the pooled batch of samples, the third nucleotide sequence having at least 80% identity to SEQ ID NO: 3.
Clause 122. The reagent kit of clause 121, wherein the first nucleotide sequence has at least 80% identity to SEQ ID NO: 1.
Clause 123. The reagent kit of clause 121 or 122, wherein the second nucleotide sequence has at least 80% identity to SEQ ID NO: 2.
Clause 124. The reagent kit of any of clauses 121-123, wherein the first amplification primer pair includes an amplification primer having a nucleotide sequence having at least 80% identity to SEQ ID NO: 4.
Clause 125. The reagent kit of any of clauses 121-124, wherein the first amplification primer pair includes an amplification primer having a nucleotide sequence having at least 80% identity to SEQ ID NO: 5.
Clause 126. The reagent kit of any of clauses 121-125, wherein the second amplification primer pair includes an amplification primer having a nucleotide sequence having at least 80% identity to SEQ ID NO: 6.
Clause 127. The reagent kit of any of clauses 121-126, wherein the second amplification primer pair includes an amplification primer having a nucleotide sequence having at least 80% identity to SEQ ID NO: 7.
Clause 128. The reagent kit of any of clauses 121-127, further comprising a first probe having a nucleotide sequence having at least 80% identity to SEQ ID NO: 8.
Clause 129. The reagent kit of any of clauses 121-128, further comprising a second probe having a nucleotide sequence having at least 80% identity to SEQ ID NO: 9.
Clause 130. The reagent kit of clause 129, wherein the first probe and the second probe have distinct labels.
Clause 131. The reagent kit of any of clauses 121-130, further comprising a third amplification primer pair for detecting presence of RNAse P gene in the pooled batch of samples.
Clause 132. The reagent kit of clause 131, wherein the third amplification primer pair includes an amplification primer having a nucleotide sequence having at least 80% identity to SEQ ID NO: 10.
Clause 133. The reagent kit of clause 131 or 132, wherein the third amplification primer pair includes an amplification primer having a nucleotide sequence having at least 80% identity to SEQ ID NO: 11.
Clause 134. The reagent kit of any of clauses 131-133, further comprising a third probe having a nucleotide sequence having at least 80% identity to SEQ ID NO: 12.
Clause 135. The reagent kit of clause 134, wherein the first probe, the second probe, and the third probe have distinct labels.
Clause 136. The reagent kit of any of clauses 121-135, wherein the reagent kit does not include any probe for determining presence of the third nucleotide sequence in the pooled batch of samples.
Clause 137. Use of the reagent kit of any of clauses 121-136 for determining absence of severe acute respiratory syndrome coronavirus 2 (SARS-CoV 2) in a pooled batch of samples.

In some embodiments, the features described with respect to clauses 1-42 are implemented in the methods and/or reagent kits described in clauses 43-137. In some embodiments, the features described with respect to clauses 43-89 are implemented in the methods and/or reagent kits described in claims 1-42 and 90-137. In some embodiments, the featuers described with respect to clauses 90-137 are implemented in the methods and/or reagent kits described in claims 1-89. For brevity, such details are not repeated herein.

Although some of various drawings illustrate a number of operations in a particular order, operations that are not order dependent may be reordered and other operations may be combined or broken out. While some reordering or other groupings are specifically mentioned, others will be obvious to those of ordinary skill in the art, so the ordering and groupings presented herein are not an exhaustive list of alternatives.

The foregoing description, for purpose of explanation, has been described with reference to specific embodiments. However, the illustrative discussions above are not intended to be exhaustive or to limit the scope of claims to the precise forms disclosed. Many modifications and variations are possible in view of the above teachings. The embodiments were chosen and described in order to best explain the principles of the various described embodiments and their practical applications, to thereby enable others skilled in the art to best utilize the principles and the various described embodiments with various modifications as are suited to the particular use contemplated.

What is claimed is:

1. A method for detecting severe acute respiratory syndrome coronavirus 2 (SARS-CoV 2) in a sample from a subject, the method comprising:
determining presence of a first nucleotide sequence or a second nucleotide sequence in the sample, wherein:
presence of SARS-CoV 2 is detected independently of determining presence of a third nucleotide sequence in the sample,
the first nucleotide sequence has at least 80% identity to SEQ ID NO: 1,
the second nucleotide sequence has at least 80% identity to SEQ ID NO: 2, and
the third nucleotide sequence has at least 80% identity to SEQ ID NO: 3.

2. The method of claim 1, wherein the presence of the first nucleotide sequence in the sample is determined using a first amplification primer pair and the first amplification primer pair includes an amplification primer having a nucleotide sequence having at least 80% identity to SEQ ID NO: 4.

3. The method of claim 1, wherein the presence of the first nucleotide sequence in the sample is determined using a first amplification primer pair and the first amplification primer pair includes an amplification primer having a nucleotide sequence having at leats 80% identity to SEQ ID NO: 5.

4. The method of claim 1, wherein the presence of the second nucleotide sequence in the sample is determined using a second amplification primer pair and the second amplification primer pair includes an amplification primer having a nucleotide sequence having at least 80% identity to SEQ ID NO: 6.

5. The method of claim 1, wherein the presence of the second nucleotide sequence in the sample is determined using a second amplification primer pair and the second amplification primer pair includes an amplification primer having a nucleotide sequence having at least 80% identity to SEQ ID NO: 7.

6. The method of claim 1, wherein the presence of the first nucleotide sequence is determined also using a first probe having a nucleotide sequence having at least 80% identity to SEQ ID NO: 8.

7. The method of claim 1, wherein the presence of the second nucleotide sequence is determined also using a second probe having a nucleotide sequence having at least 80% identity to SEQ ID NO: 9.

8. The method of claim 1, further comprising:
determining presence of RNAse P gene in the sample using a third amplification primer pair.

9. The method of claim 8, wherein the third amplification primer pair includes an amplification primer having a nucleotide sequence having at least 80% identity to SEQ ID NO: 10.

10. The method of claim 8, wherein the third amplification primer pair includes an amplification primer having a nucleotide sequence having at least 80% identity to SEQ ID NO: 8.

11. The method of claim 8, wherein presence of RNAse P gene in the sample is determined also using a third probe having a nucleotide sequence having at least 80% identity to SEQ ID NO: 9.

12. The method of claim 8, wherein the presence of RNAse P gene is detected by real-time reverse transcription polymerase chain reaction.

13. The method of claim 8, wherein the RNAse P gene is human RNAse P gene.

14. The method of claim 1, wherein the presence of the first nucleotide sequence is detected by real-time reverse transcription polymerase chain reaction.

15. The method of claim 1, wherein the presence of the second nucleotide sequence is detected by real-time reverse transcription polymerase chain reaction.

16. The method of claim 1, wherein the subject is a human.

17. A method for detecting severe acute respiratory syndrome coronavirus 2 (SARS-CoV 2) in a sample from a subject, the method comprising:
determining that SARS-CoV 2 is present in the sample in accordance with at least a determination that a first nucleotide sequence or a second nucleotide sequence is present in the sample independent of whether a third nucleotide sequence is present in the sample, wherein:
the first nucleotide sequence has at least 80% identity to SEQ ID NO: 1,
the second nucleotide sequence has at least 80% identity to SEQ ID NO: 2, and
the third nucleotide sequence has at least 80% identity to SEQ ID NO: 3.

18. A reagent kit, comprising:
a first amplification primer pair for determining presence of a first nucleotide sequence in a sample or a second amplification primer pair for determining presence of a second nucleotide sequence in the sample,
wherein the reagent kit does not include an amplification primer pair for determining presence of a third nucleotide sequence in the sample, the third nucleotide sequence having at least 80% identity to SEQ ID NO: 3.

19. The reagent kit of claim 18, wherein the first nucleotide sequence has at least 80% identity to SEQ ID NO: 1.

20. The reagent kit of claim 18, wherein the second nucleotide sequence has at least 80% identity to SEQ ID NO: 2.
